(19) United States
(12) Patent Application Publication
(10) Pub. No.: US 2008/0319560 A1
(43) Pub. Date: Dec. 25, 2008

(54) AUTOMATED RECIPE COMPOSER

(76) Inventor: BADR FAYEZ, Jeddah (SA)

Correspondence Address: MICHAEL RIES, 318 PARKER PLACE, OSWEGO, IL, 60543, US

(21) Appl. No.: 12/137,549

(22) Filed: Jun. 12, 2008

Related U.S. Application Data
(60) Provisional application No. 60/945,082, filed on Jun. 19, 2007.

(51) Int. Cl. G01N 33/02; H05B 1/02; G06F 17/00; G09B 19/00

(52) U.S. Cl. 700/90; 99/325; 219/490; 434/127; 426/231

(57) ABSTRACT

Disclosed in the present invention is a way to automatically combine a recipe with an automated recipe composer.

This application claims priority to U.S. Provisional Application 60/945,082 filed Jun. 19, 2007, the entire disclosure of which is incorporated by reference.

### TECHNICAL FIELD AND BACKGROUND

The present invention relates to a way to automatically combine a recipe. More specifically, the present invention provides an automated recipe composer.

The present invention is a burner that doubles up as a scale, An infra red thermometer which measures the temperature of the cooking vessel, a dial to control the flame, a touch screen panel that permits the chef to specify the kind of ingredient used in the cooking process, USB ports to connect the external scale used to mix items that does not need any cooking. The present invention is also used to connect to a computer which enters the information recoded by the device in word format to produce a final recipe with method of preparation.

### DETAILED DESCRIPTION

Various operations will be described as multiple discrete operations, in turn, in a manner that is most helpful in understanding the present invention, however, the order of description should not be construed as to imply that these operations are necessarily order dependent. In particular, these operations need not be performed in the order of presentation.

The phrase “in one embodiment” is used repeatedly. The phrase generally does not refer to the same embodiment, however, it may. The terms “comprising”, “having” and “including” are synonymous, unless the context dictates otherwise.

The present invention is used by step 1 turning on a machine by pressing the power button marked 1/0. Step 2 Pressing a compose button. Step 3 if the items used for this recipe are known and the order of which they will be added then skip to step 9. Step 4 Use the vessel that is required and then press the vessel button. Step 5 you can record the vessel type and weight by pressing on an empty vessel position. Step 6 This will allow you to record the name and brief description of the vessel using the key pad or by entering via the computer. Step 7 the vessel's weight will be automatically recorded. Step 8 Turn on the heat then add the ingredients one by one after identifying it on the touch screen panel marked by either: meat, poultry, fish, vegetables, fruits, spices, liquids, dry, or wet ingredients.

a. Each button has a subcategory that specifically identifies the ingredient used.

b. Ingredients can be added by specifying its category or subcategory and pressing the add button.

c. This will allow you to specify the ingredient and whether it is made from a combination of other ingredients and adding those details to it.

Step 9 you can preset the order of ingredients that will be added in advance by assigning each numbered position of the recipe on the composition screen with an ingredient. That way you can simply press the down button after the addition of the preceding ingredient. Step 10 the software within the machine will record all the ingredients used, their weights, the time that each one has been added when the composition started, the heat of the vessel at the time of every ingredient added, the heat dial adjustments, and the duration of the entire recipe.

While the present invention has been related in terms of the foregoing embodiments, those skilled in the art will recognize that the invention is not limited to the embodiments described. The present invention can be practiced with modification and alteration within the spirit and scope of the appended claims. Thus, the description is to be regarded as illustrative instead of restrictive on the present invention.

What is claimed is:

1. A device comprising:
a burner that doubles up as a scale;
an infra red thermometer which measures the temperature of the cooking vessel;
a dial to control the flame;
a touch screen panel that permits a chef to specify a kind of ingredient used in the cooking process; and
USB ports to connect an external scale used to mix items that does not need any cooking and connect to a computer which enters information recoded by the device in word format to produce a final recipe with a method of preparation.

2. A method comprising:
turning on a machine by pressing the power button marked 1/0;
pressing a compose button on the machine;
using a vessel that is required and then press the vessel button on the machine;
recording the vessel type and weight by pressing on an empty vessel position;
recording a name and brief description of the vessel using a selected one of a key pad and by entering via a computer;
automatically recording the vessel's weight;
turning on heat then adding ingredients one by one after identifying it on the touch screen panel marked by either meat, poultry, fish, vegetables, fruits, spices, liquids, dry, or wet ingredients, each button has a subcategory that specifically identifies the ingredient used, ingredients can be added by specifying its category or subcategory and pressing the add button, this will allow you to specify the ingredient and whether it is made from a combination of other ingredients and adding those details to it;
presetting the order of ingredients that will be added in advance by assigning each numbered position of the recipe on the composition screen with an ingredient, that way you can simply press the down button after the addition of the preceding ingredient; and
recording within the machine with software all the ingredients used, their weights, the time that each one has been added when the composition started, the heat of the vessel at the time of every ingredient added, the heat dial adjustments, and the duration of the entire recipe.

3. A method comprising:
turning on a machine by pressing the power button marked 1/0;
pressing a compose button on the machine;
presetting the order of ingredients that will be added in advance by assigning each numbered position of the recipe on the composition screen with an ingredient, that way you can simply press the down button after the addition of the preceding ingredient; and
recording within the machine with software all the ingredients used, their weights, the time that each one has been added when the composition started, the heat of the vessel at the time of every ingredient added, the heat dial adjustments, and the duration of the entire recipe.
